(19) United States
(12) Patent Application Publication
(10) Pub. No.: US 2024/0092969 A1
(43) Pub. Date: Mar. 21, 2024

(54) FUNCTIONALISED BIODEGRADABLE POLYESTER POLYMERS

(71) Applicant: UEA Enterprises Limited, Norwich (GB)

(72) Inventor: Aram Omer SAEED, Norwich (GB)

(21) Appl. No.: 18/548,696

(22) PCT Filed: Mar. 7, 2022
(86) PCT No.: PCT/GB2022/050595

§ 371 Date: Sep. 1, 2023

(30) Foreign Application Priority Data
Mar. 5, 2021 (GB) 2103120.8

(51) Int. Cl. C08G 63/685; A61L 27/18; A61L 27/58; C08G 63/08; C08G 63/82; C08G 63/85

(52) U.S. Cl. CPC C08G 63/685; A61L 27/18; A61L 27/58; C08G 63/08; C08G 63/823; C08G 63/85; C08G 2230/00

(57) ABSTRACT

Described herein is a biodegradable polymer comprising an amine-terminated polyester polymer. Also described herein are microparticles comprising the biodegradable polymer; a method of producing the biodegradable polymer comprising initiating a ring-opening polymerisation of a cyclic ester with an amino alcohol initiator; and a variety of uses for the biodegradable polymer, including in tissue engineering and regenerative medicine, for example, as a microcarrier for biologics.

### FIELD OF THE INVENTION

This invention is directed to functionalised biodegradable polyester polymers for biomedical applications, including but not limited to microparticles for cell culture, cell delivery, delivery of biologics, vaccines, and implants.

### BACKGROUND OF THE INVENTION

Synthetic hydrolytically degradable polyesters, such as poly(lactic acid) (PLA), poly(glycolic acid) (PGA), poly(lactic-co-glycolic acid) (PLGA), and poly(caprolactone) (PCL), are one of the most widely used classes of polymer in the biomedical field, with applications including but not limited to cell culture systems, drug delivery systems, tissue repair scaffolds and medical devices. Their biologically inert nature and tuneable degradation profile coupled with their straight-forward and cost-effective production makes these polymers appealing both for research and commercial usage. Microparticles (MPs) are one such use of these polyester polymers. Despite the success of the polyester polymers and associated microparticles, their applications in the development of more advanced tissue culture and cell delivery platforms have been hampered by their lack of cell adhesion moieties, which is largely due to the hydrophobic surface of these polymers. Consequently, the development of strategies to modify the surface of these polymers has garnered significant interest.

Several physical and chemical modification techniques have been established to address these issues. Examples of physical modifications included blending or surface coating of the polymers with natural polymers that contain cell adhesive sites (e.g., laminin, fibronectin, and vitronectin). In regard to chemical modifications, a popular strategy is the process of aminolysis to generate an active site on the polymer surface. This active site acts as a target for subsequent activation and conjugation with small molecules such as arginine-glycine-aspartic acid (RGD) peptides. More recently, advanced surface-grafting of cationic polymers to a preformed polyester was reported by using a specialist chain-transfer molecule and living polymerization techniques. Despite the progress in the development of post-modification techniques the nature of these methods remains complex, challenging, and troublesome for translational medicine.

Additionally, PCL microparticles pre-functionalized with azide functional groups have been created for subsequent reaction of with a pre-functionalized model protein through copper-free click chemistry of the azide functional groups with an alkyne in the model protein.

The present inventors have found that examples of the invention as described herein avoid or at least mitigate at least one of the difficulties described above.

### SUMMARY

In an aspect, there is provided a biodegradable polymer having the following formula:

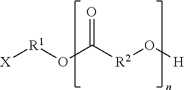

wherein
X is selected from NH², NHR³, NR³₂ and NR³₃⁺;
R¹ is an alkylene group;
each R² is independently selected from a —(CR⁴₂)ₘ— group or a —(CR⁴₂)ₒO(CR⁴₂)ₚ— group;
each R³ is independently an alkyl group;
each R⁴ is independently selected from hydrogen and an alkyl group; and
n, m, o and p are independently 1 or more.

In another aspect, there is provided microparticles comprising the biodegradable polymer.

In a further aspect, there is provided a method of producing the biodegradable polymer comprising:
combining an amino alcohol with a cyclic ester and performing ring-opening polymerisation to form a biodegradable polymer;
wherein the amino alcohol has the formula XR¹OH; and
wherein
the cyclic ester is selected from cyclic monoesters having the formula (1), cyclic diesters having the formula (2) and combinations thereof;

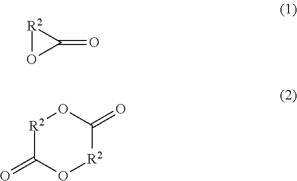

X is selected from NH², NHR³, NR³₂ and NR³₃⁺;
R¹ is an alkylene group;
each R² is independently selected from a —(CR⁴₂)ₘ— group or a —(CR⁴₂)ₒO(CR⁴₂)ₚ— group;
each R³ is independently an alkyl group;
each R⁴ is independently selected from hydrogen and an alkyl group; and
m, o and p are independently 1 or more.

In another aspect, there is provided a method of culturing cells comprising combining the biodegradable polymer or the microparticles with a cell culture medium and incubating the composition.

In another aspect, there is provided a composition comprising cells supported on the biodegradable polymer or on the microparticles.

In a further aspect, there is provided a supported tissue structure comprising cells supported on the biodegradable polymer or the microparticles.

In another aspect, there is provided a tissue scaffold, a microcarrier for of biologics or a medical implant comprising the biodegradable polymer or the microparticles.

In another aspect, there is provided the use of the biodegradable polymer or the microparticles in tissue engineering or regenerative medicine.

The present invention introduces a new and practical methodology for surface modification of polyester based materials that can readily promote cell adhesion at physiological conditions. This strategy allows the insertion of cell adhesion moieties such as small cationic amino functional groups into the structure of the polyester polymers in a one-step process. These cationic groups enable interaction and adhesion of the polyester polymer with polyanionic cell membranes without requiring any post-functionalization method.

As an example of this method, lactide and glycolic monomers were co-polymerized in the presence of 2-dimethylaminoethanol (a tertiary amine) (DMAEtOH) as a ring-opening polymerisation (ROP) initiator to generate a PLGA_{DMAE} polymer with terminal dimethylaminoethyl (DMAE) groups. Subsequently, PLGA_{DMAE} microparticles (MPs) were fabricated via membrane emulsification, allowing control of the particle size and particle size distribution. Surface adhesion of adipose tissue-derived stem cells (ADSCs) to the PLGA_{DMAE} MPs was analysed and compared with that of unmodified PLGA MPs under the same cell culture conditions.

Without wishing to be bound by theory, it is believed that since the pKa value of DMAEtOH is higher than physiological pH (and thus it is expected to be positively charged), interaction of the pre-functionalized polyester polymer with polyanionic cell membranes is promoted. Moreover, despite the change in the terminal functional groups, no detrimental effect on the bulk properties, cytotoxicity and degradation profile of the polymers was found.

This new strategy of one-step pre-functionalization of polyester polymers, exemplified via PLGA_{DMAE} synthesis using a bulk ROP process to introduce cell adhesion moieties, resulted in MPs with surfaces that promote cell attachment under physiological conditions, which can be considered as a significant step forward. This new method can address the shortcomings, namely, the lack of cell adhesion, associated with the polyester polymers for advanced therapeutics and culture system applications.

Furthermore, the biodegradable nature of such polymers also means that after cell expansion in the presence of the microparticles, no cell harvesting is required because the biodegradable polymer can be administered together with the cells.

### BRIEF DESCRIPTION OF THE FIGURES

FIG. 1 is a schematic illustration of the synthesis and fabrication of the pre-functionalised Example (PLGA_{DMAE}) and the Comparative (PLGA) microparticles. A) One-step ring-opening polymerisation (ROP) of D,L-lactide and glycolide monomers by using a Tin(II) catalyst and heating at 140° C. for 48 h under nitrogen. B) Fabrication of the microparticles (MPs) via the membrane emulsification process.

FIG. 2 is a graph of the A) thermogravimetric analysis of PLGA (--) and PLGA_{DMAE} polymers (▪▪), depicting percentage weight loss (%) as a function of temperature (° C.) (top) and derivative weight (%/° C.) as a function of temperature (° C.) (bottom) and B) Differential Scanning Calorimetry thermograms obtained for PLGA (--) and PLGA_{DMAE} polymers (▪▪), displaying the heat flow (W/G) as a function of temperature, and including the T_{g} onset, midpoint, and endpoint regions. DSC thermograms were obtained from the second heating cycle.

FIG. 3 shows that there are no significant differences in the morphology or size distribution of the PLGA and PLGA_{DMAE} Microparticles. A) Phase contrast imaging of freshly prepared (left) and solidified (right) PLGA and PLGA_{DMAE} microparticles (scale bar is 100 μm (10× magnification)). B) SEM images of freeze-dried PLGA and PLGA_{DMAE} microparticles (scale bar is 100 μm). C) Frequency histograms, Span values and accompanying information regarding the size distribution and mean particle size comparison of PLGA and PLGA_{DMAE} microparticles. Mean particle size comparison between PLGA and PLGA_{DMAE} microparticles is shown as bar charts. Data is presented as mean particle size (μm)±SD (n=20 representative images). A two-tailed unpaired t test statistical analysis was applied (Ns: non-significant).

FIG. 4 shows that ADSC cell adhesion can be enhanced via Method 2, which has been developed to optimise microparticle and ADSC attachment. A) Fluorescent imaging of cell nuclei via Hoechst staining (grey), corresponding phase contrast view and merges of nuclei staining and phase contrast for ADSCs mixed with PLGA_{DMAE} MPs via method 1 (standard cell and MP mixing) and method 2, respectively. B) Quantification of the number of Hoechst-stained nuclei per field of review. Data are presented as an average of Hoechst-stained nuclei counted per field (OD)±SD (n=3 independent cell culture replicates) in three random locations, therefore using a total of 9 images per sample. A two-tailed unpaired t test statistical analysis was applied (****p<0.0001).

FIG. 5 shows that PLGA_{DMAE} microparticles retain a low cytotoxicity profile that is comparable to un-functionalised PLGA microparticles. A) LDH assay performed on ADSCs in the presence of PLGA or PLGA_{DMAE} MPs, analysed at days 1, 5, 7, and 14 post-culture. Data are presented as optical density values (OD)±SD (n=3 independent cell culture replicates). Differences between groups were assessed via a two-way ANOVA with a Tukey's multiple comparison test (Ns: non-significant, *p=0.03, ***p<0.0002, ****p<0.0001. B) Representative fluorescent images showing the LIVE (Calcein AM- green)/DEAD (ethidium homodimer-1 red) staining of the co-cultured cells with the MPs at different time intervals (scale bar is 100 μm; 20× magnification). C) Average number of cells per field of view that were classified as either alive or dead. Data are presented as the average of counted cells per field±SD (n=3 independent technical cell culture replicates) in three random locations, and thus using a total of 9 images per sample (Ns: non-significant).

FIG. 6 shows that ADSCs readily adhere to PLGA_{DMAE} microparticles and show differential cellular morphologies when compared to ADSCs co-cultured with unmodified PLGA microparticles. Representative SEM images of ADSCs co-cultured with PLGA microparticles (top) or PLGA_{DMAE} microparticles (bottom; all scale bars are 100 μm).

FIG. 7 shows that PLGA_{DMAE} microparticles degrade more rapidly and uniformly than unmodified PLGA microparticles. A) SEM images of PLGA and PLGA_{DMAE} microparticles immersed in PBS at 37° C. on days 7, 14, and 21 after incubation (scale bars are 100 μm (high magnification) or 10 μm (low magnification)). B) Normalised GPC chromatograms, recorded with a Refractive Index detector, depicting the shift in the molecular weight over time for i) PLGA and ii) PLGA_{DMAE} MPs.

FIG. 8 shows the morphology and size distribution of larger PLGA_{DMAE} microparticles. Phase contrast imaging of A) freshly prepared and B) solidified PGLA_{DMAE} microparticles (scale bar is 100 μm (10× magnification). C) SEM images of freeze-dried PLGA_{DMAE} microparticles (scale bar is 100 μm). D) Frequency histograms and Span values depicting the size distribution and mean particle size of PLGA_{DMAE} microparticles.

FIG. 9 shows cell attachment to PGLA_{DMAE} microparticles in controlled suspension conditions. Phase contrast imaging of human adipose-derived stem cells (ADSCs) attached to PGLA_{DMAE} microparticles 24 hours after culture (scale bar is 100 μm; A) 10× magnification and B) C) and D) 20× magnification). Black arrows indicate the presence of cells attached to the PLGA_{DMAE} microparticles.

### DETAILED DESCRIPTION

Before the present disclosure is disclosed and described, it is to be understood that this disclosure is not limited to the particular process steps and materials disclosed herein because such process steps and materials may vary somewhat. It is also to be understood that the terminology used herein is used for the purpose of describing particular embodiments. The terms are not intended to be limiting because the scope is intended to be limited by the appended claims and equivalents thereof.

It is noted that, as used in this specification and the appended claims, the singular forms “a,” “an,” and “the” include plural referents unless the context clearly dictates otherwise.

As used herein, “co-polymer” refers to a polymer that is polymerized from at least two monomers.

As used herein, “alkyl”, or similar expressions such as “alk” in alkoxy, may refer to a branched, unbranched, or cyclic saturated hydrocarbon, which may, in some examples, contain from 1 to about 50 carbon atoms, or 1 to about 40 carbon atoms, or 1 to about 30 carbon atoms, or 1 to about 10 carbon atoms, or 1 to about 5 carbon atoms.

As used herein, “alkylene” may refer to a divalent group derived by removal of two hydrogen atoms from one carbon atom or one hydrogen atom from two different carbon atoms of an alkane, which may, in some examples, contain from 1 to about 50 carbon atoms, or 1 to about 40 carbon atoms, or 1 to about 30 carbon atoms, or 1 to about 10 carbon atoms, or 1 to about 5 carbon atoms.

A certain monomer may be described herein as constituting a certain weight percentage of a polymer. This indicates that the repeating units formed from the said monomer in the polymer constitute said weight percentage of the polymer.

If a standard test is mentioned herein, unless otherwise stated, the version of the test to be referred to is the most recent at the time of filing this patent application.

As used herein, the term “about” is used to provide flexibility to a numerical range endpoint by providing that a given value may be a little above or a little below the endpoint to allow for variation in test methods or apparatus. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not just the individual numerical values or sub-ranges encompassed within that range as if each numerical value and sub-range is explicitly recited. As an illustration, a numerical range of “about 1 wt. % to about 5 wt. %” should be interpreted to include not just the explicitly recited values of about 1 wt. % to about 5 wt. %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting a single numerical value. Furthermore, such an interpretation should apply regardless of the breadth of the range or the characteristics being described.

As used herein, unless otherwise stated, wt. % values are to be taken as referring to a weight-for-weight (w/w) percentage of solids in the ink composition, and not including the weight of any fluid present.

Unless otherwise stated, any feature described herein can be combined with any aspect or any other feature described herein.

### Biodegradable Polymer

In an aspect, there is provided a biodegradable polymer comprising an amine-terminated polyester polymer. In some examples, the biodegradable polymer may have the following formula:

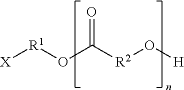

wherein X is selected from NH₂, NHR³, NR³₂ and NR³₃⁺; R¹ is an alkylene group; each R² is independently selected from a —(CR⁴₂)ₘ— group or a —(CR⁴₂)ₒO(CR⁴₂)ₚ— group; each R³ is independently an alkyl group; each R⁴ is independently selected from hydrogen and an alkyl group; and m, o and p are independently 1 or more. In some examples, the biodegradable polymer may have the following formula:

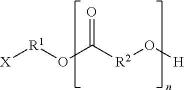

wherein X is selected from NH₂, NHR³, NR³₂ and NR³₃⁺; R¹ is an alkylene group; each R² is independently selected from a —(CR⁴₂)ₘ— group or a —(CR⁴₂)ₒO(CR⁴₂)ₚ— group; each R³ is independently an alkyl group; each R⁴ is independently selected from hydrogen and an alkyl group; and n, m, o and p are independently 1 or more.

R¹ is an alkylene group. In some examples, R¹ is any alkylene group, for example, a branched, unbranched or cyclic alkylene group. In some examples, R¹ is a branched or unbranched alkylene group. In some examples, R¹ is a branched alkylene group. In some examples, R¹ is an unbranched alkylene group.

In some examples, R¹ is a C1 to C20 alkylene group, for example, a C1to C10 alkylene group, or a C1 to C5 alkylene group. In some examples, R¹ is selected from methylene, ethylene, methyl methylene (—CH(CH₃)—), propylene, methyl ethylene (e.g., —CH(CH₃)CH₂— or —CH₂CH(CH₃)—), ethyl methylene (—CH(CH₂CH₃)—), butylene, methyl propylene (e.g., —CH(CH₃)CH₂CH₂—, —CH₂CH(CH₃)CH₂—, or —CH₂CH₂CH(CH₃)—), ethyl ethylene (e.g., —CH(CH₂CH₃)CH₂— or —CH₂CH(CH₂CH₃)—), propyl methylene (e.g., —CH—(CH₂CH₂CH₃)— or —CH[CH(CH₃)(CH₃)]—, pentylene, methyl butylene (e.g., —CH(CH₃)CH₂—CH₂CH₂—, —CH₂CH(CH₃)CH₂CH₂—, —CH₂CH₂CH(CH₃)CH₂—, or —CH₂CH₂CH₂CH(CH₃)—), ethyl propylene (e.g., —CH(CH₂CH₃)CH₂CH₂—, —CH₂CH(CH₂CH₃)CH₂—, or —CH₂CH₂CH—(CH₂CH₃)—), propyl ethylene (e.g., —CH(CH₂CH₂CH₃)CH₂—, —CH[CH(CH₃)(CH₃)]CH₂—, —CH₂CH(CH₂CH₂CH₃)—, —CH₂CH[CH(CH₃)(CH₃)]—) or butyl methylene (e.g., —CH(CH₂—CH₂CH₂CH₃)—, —CH[CH(CH₃)CH₂CH₃]—, —CH[CH₂CH(CH₃)(CH₃)]— or —CH[C(CH₂CH₃)—(CH₃)]). In some examples, R¹ is selected from methylene, ethylene, propylene, butylene, or pentylene. In some examples, R¹ is ethylene (i.e. —CH₂CH₂—).

Each R² is independently selected from a —(CR⁴₂)ₘ— group or a —(CR⁴₂)ₒO(CR⁴₂)ₚ— group. In some examples, each R² is independently a —(CR⁴₂)ₘ— group. In some examples, m is 1 or more, for example, 1 to 20, 1 to 10, 1 to 5, or 1, 2, 3, 4 or 5. In some examples, o is 1 or more, for example, 1 to 20, 1 to 10, 1 to 5, or 1, 2, 3, 4 or 5. In some examples, p is 1 or more, for example, 1 to 20, 1 to 10, 1 to 5, or 1, 2, 3, 4 or 5. In some examples, o+p is 2 or more, for example, 2 to 20, 2 to 10, 2 to 5, 2, 3, 4, or 5. In some examples, o is 1 and p is 2.

Each R⁴ is independently selected from hydrogen and an alkyl group. In some examples, each R⁴ is independently selected from hydrogen and a C1 to C20 alkyl group, for example, a C1 to C10 alkyl group, C1 to C5 alkyl group, a C1 alkyl group, a C2 alkyl group, a C3 alkyl group, a C4 alkyl group or a C5 allkyl group. In some examples, R⁴ is independently selected from hydrogen, methyl, ethyl, propyl, isopropyl, butyl, sec-butyl, isobutyl, tert-butyl, or pentyl. In some examples, each R⁴ is independently selected from hydrogen and a C1 to C20 alkyl group, for example, C1 to C10, or C1 to C5; and m is selected from 1 to 20, for example, 1 to 10 or 1 to 5.

In some examples, R² is —CH₂—, —CH(CH₃)—, —(CH₂)₅—, —CH₂CH(CH₃)—, —CH₂CH(CH₂CH₃)— or —CH₂OCH₂CH₂—.

X is selected from NH₂, NHR³, NR³₂ and NR³₃⁺. In some examples, X is selected from NHR³ and NR³₂. In some examples, X is NH₂. In some examples, X is NHR³. In some examples, X is NR³₂. In some examples, X is NR³₃⁺. In some examples, X has a cationic charge at physiological pH or has a permanent cationic charge (i.e., is an —NR³₃⁺ group). In some examples, physiological pH is a pH of 7 to 8, for example, 7.4.

Each R³ is independently an alkyl group. In some examples, each R³ is independently a C1 to C20 alkyl group, for example, a C1 to C10 alkyl group, a C1 to C5, for example, methyl, ethyl, propyl, or butyl.

In some examples, X is selected from NH₂, NHMe, NMe₂, NMe₃⁺, NHEt, NEt₂, NEt₃⁺, NMeEt, NMe₂Et⁺, NEt₂Me, NHPr, NPr₂, NPr₃⁺, NMePr, NEtPr, NMe₂Pr⁺, NMePr₂⁺, NEt₂Pr⁺, NEtPr₂⁺, NH(iPr), N(iPr)₂, N(iPr)₃, NMe(iPr), NEt(iPr), NPr(iPr), NMe₂(iPr)⁺, NMe(iPr)₂⁺, NEt(iPr)₂⁺, NPr₂(iPr)⁺, NPr(iPr)₂⁺. In some examples, X is selected from NH₂, NHMe, NMe₂, and NMe₃⁺. In some examples, X is NMe₂.

In some examples, R¹ is a C1 to C5 alkylene group; R² is —(CR⁴)ₘ— or —(CR⁴₂)ₒO(CR⁴₂)ₚ—; each R³ is independently a C1 to C5 alkyl group; each R⁴ is independently hydrogen or a C1 to C5 alkyl group; and m is selected from 1 to 5, o is selected from 1 to 5 and p is selected from 1 to 5. In some examples, R¹ is a C1 to C5 alkylene group; R² is —(CR⁴)ₘ— or —(CR⁴₂)ₒO(CR⁴₂)ₚ—; each R³ is independently a C1 to C5 alkyl group; each R⁴ is independently hydrogen or a C1 to C5 alkyl group; and m is selected from 1 to 5, o is selected from 1 or 2 and p is selected from 1 or 2; and optionally, wherein o+p is 3. In some examples, R¹ is a C1 to C5 alkylene group; R² is —(CR⁴)ₘ— each R³ is independently a C1 to C5 alkyl group; each R⁴ is independently hydrogen or a C1 to C5 alkyl group; and m is selected from 1 to 5. In some examples, R¹ is a C1 to C5 alkylene group; each R³ is independently a C1 to C5 alkyl group; each R⁴ is independently hydrogen or a C1 to C5 alkyl group; and m is selected from 1 to 5.

In some examples, XR¹— is selected from 2-(methylamino)ethyl-, 2-(dimethylamino)ethyl-, 2-(methylamino)methyl-, 2-(dimethylamino)methyl-, 2-(methylamino)propyl-, 2-(dimethylamino) propyl-, 2-(methylamino)butyl-, 2-(dimethylamino)butyl-, 2-(ethylamino)ethyl-, 2-(diethylamino)ethyl-, 2-(methylethylamino)ethyl-, 2-(ethyl-amino)methyl-, 2-(diethylamino)methyl-, 2-(methylethylamino)methyl-, 2-(ethylamino)-propyl, 2-(diethylamino)propyl-, 2-(methylethylamino)propyl-, 2-(ethylamino)butyl-, 2-(diethylamino)butyl-, 2-(methylethylamino)butyl- and combinations thereof.

In some examples, the biodegradable polymer may be a polymer or a co-polymer. In some examples, the biodegradable polymer may be a co-polymer of two or more monomers, for example, two monomers.

In some examples, biodegradable polymer may comprise an amine-terminated polyester. The amine-terminated polyester may comprise an amine-terminated poly(lactic acid), an amine-terminated poly(glycolic acid), an amine-terminated poly(lactic-co-glycolic acid), an amine-terminated poly(caprolactone), an amine-terminated poly(hydroxybutyrate), an amine-terminated poly(p-dioxanone), an amine-terminated poly(3-hydroxyvalerate), or a mixture thereof. In some examples, the biodegradable polymer may comprise an XR¹O-terminated poly(lactic acid), an XR¹O-terminated poly(glycolic acid), an XR¹O-terminated poly(lactic-co-glycolic acid), an XR¹O-terminated poly(caprolactone), an XR¹O-terminated poly(hydroxybutyrate), an XR¹O-terminated poly(p-dioxanone), or an XR¹O-terminated poly(3-hydroxyvalerate).

n is 1 or more. In some examples, n is an integer. In some examples, n is any integer such that the biodegradable polymer has a weight average molecular weight (M_{w}) is as defined herein. In some examples, n is any integer such that the biodegradable polymer has a number average molecular weight (Mₙ) as defined herein. In some examples, n is any integer such that the biodegradable polymer has a dispersity (M_{w}/Mₙ) as defined herein. In some examples, n is any integer such that the biodegradable polymer has an M_{w}, an Mₙ and/or a dispersity (M_{w}/Mₙ) as defined herein. In some examples, n is 15 or more, for example, 20 or more or 50 or more.

In some examples, the biodegradable polymer has a weight average molecular weight (M_{w}) of at least about 1000, for example, at least about 5000, at least about 10,000, at least about 15,000, at least about 20,000, at least about 25,000, at least about 30,000, at least about 35,000, at least about 40,000, at least about 45,000, at least about 50,000, at least about 55,000, at least about 60,000, at least about 65,000, at least about 70,000, at least about 75,000, at least about 100,000, at least about 150,000, at least about 200,000, at least about 300,000, at least about 500,000, at least about 750,000, or at least about 1 million. In some examples, the biodegradable polymer has a weight average molecular weight of up to about 1 million, for example, up to about 750,000, up to about 500,000, up to about 300,000, up to about 200,000, up to about 150,000, up to about 100,000, up to about 75,000, up to about 70,000, up to about 65,000, up to about 60,000, up to about 55,000, up to about 50,000, up to about 45,000, up to about 40,000, up to about 35,000, up to about 30,000, up to about 25,000, up to about 20,000, up to about 15,000, up to about 10,000, or up to about 1000. In some examples, the biodegradable polymer has a weight average molecular weight of from about 1000 to about 1 million, for example, from about 10,000, to about 750,000, from about 10,000 to about 200,000, from about 15,000 to about 150,000, from about 15,000 to about 100,000, from about 20,000 to about 75,000, from about 20,000 to about 70,000, from about 20,000 to about 65,000, from about 25,000 to about 60,000, from about 30,000 to about 55,000, from about 35,000 to about 50,000, or from about 40,000 to about 45,000. In some examples, the weight average molecular weight was measured by using gel permeation chromatography (GPC). In some examples, the gel permeation chromatography machine is calibrated with a poly(methyl methacrylate) calibration standard (for example, with a molar mass range of 3×10⁵-6×10⁵ Da); the biodegradable polymer was dissolved in THF and the measurements were performed at 30° C.

In some examples, the biodegradable polymer has a number average molecular weight (Mₙ) of at least about 500, for example, at least about 1000, at least about 5000, at least about 10,000, at least about 15,000, at least about 20,000, at least about 25,000, at least about 30,000, at least about 35,000, at least about 40,000, at least about 45,000, at least about 50,000, at least about 100,000, at least about 200,000, or at least about 1 million. In some examples, the biodegradable polymer has a number average molecular weight of up to about 1 million, for example, up to about 200,000, up to about 100,000, up to about 50,000, up to about 45,000, up to about 40,000, up to about 35,000, up to about 30,000, up to about 25,000, up to about 20,000, up to about 15,000, up to about 10,000, up to about 5000, up to about 1000, or up to about 500. In some examples, the biodegradable polymer has a number average molecular weight of from about 500 to about 1 million, for example, about 500 to 200,000, about 1000 to about 100,000, about 500 to about 50,000, about 1000 to about 45,000, about 5000 to about 40,000, about 10,000 to about 35,000, about 15,000 to about 30,000, about 20,000 to about 25,000. In some examples, the number average molecular weight was measured by gel permeation chromatograph (GPC). In some examples, the gel permeation chromatography machine is calibrated with a poly(methyl methacrylate) calibration standard (for example, with a molar mass range of 3×10⁵-6×10⁵ Da); the biodegradable polymer was dissolved in THF and the measurements were performed at 30° C.

In some examples, the biodegradable polymer may have a dispersity (M_{w}/Mₙ) of at least about 1, for example, at least about 1.1, at least about 1.2, at least about 1.3, at least about 1.4, at least about 1.5, at least about 1.6, at least about 1.7, at least about 1.8, at least about 1.9, at least about 2, at least about 2.5, at least about 3, at least about 4. In some examples, the biodegradable polymer may have a dispersity of up to about 4, for example, up to about 3.5, up to about 3, up to about 2.5, up to about 2, up to about 1.9, up to about 1.8, up to about 1.7, up to about 1.6, up to about 1.5, up to about 1.4, up to about 1.3, up to about 1.2, up to about 1.1, up to about 1. In some examples, the biodegradable polymer may have a dispersity of from about 1 to about 4, for example, from about 1 to about 3.5, from about 1 to about 3, from about 1.1 to about 3.5, from about 1.1 to about 3, from about 1.1 to about 2.5, from about 1.1 to about 2, from about 1.2 to about 1.9, from about 1.3 to about 1.8, from about 1.4 to about 1.7, from about 1.5 to about 1.6.

In some examples, the biodegradable polymer is selected from polymers with the following formulae:

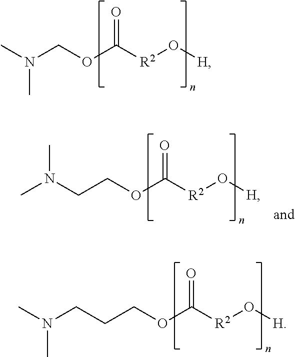

In some examples, the biodegradable polymer is selected from polymers with the following formulae:

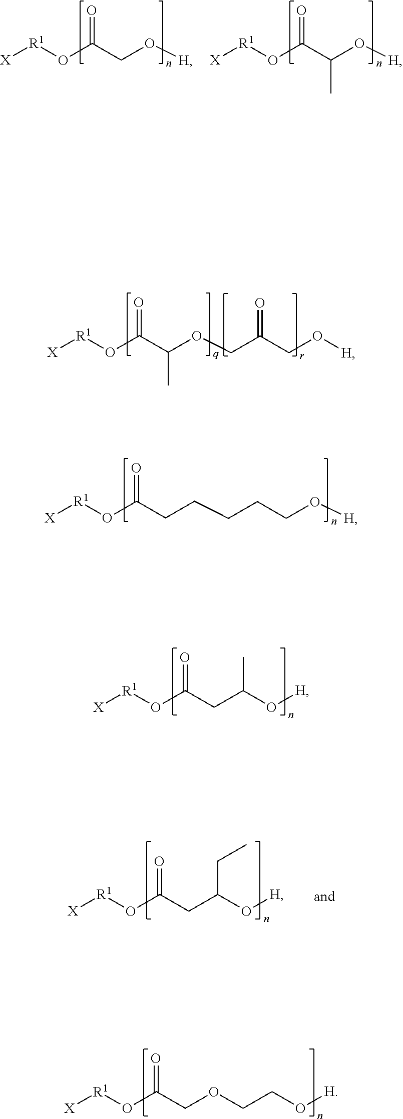

In some examples, q and r are each independently integers. In some examples, q and r may independently be n. In some examples, q+r is equal to n.

In some examples, the biodegradable polymer may be selected from polymers with the following formulae:

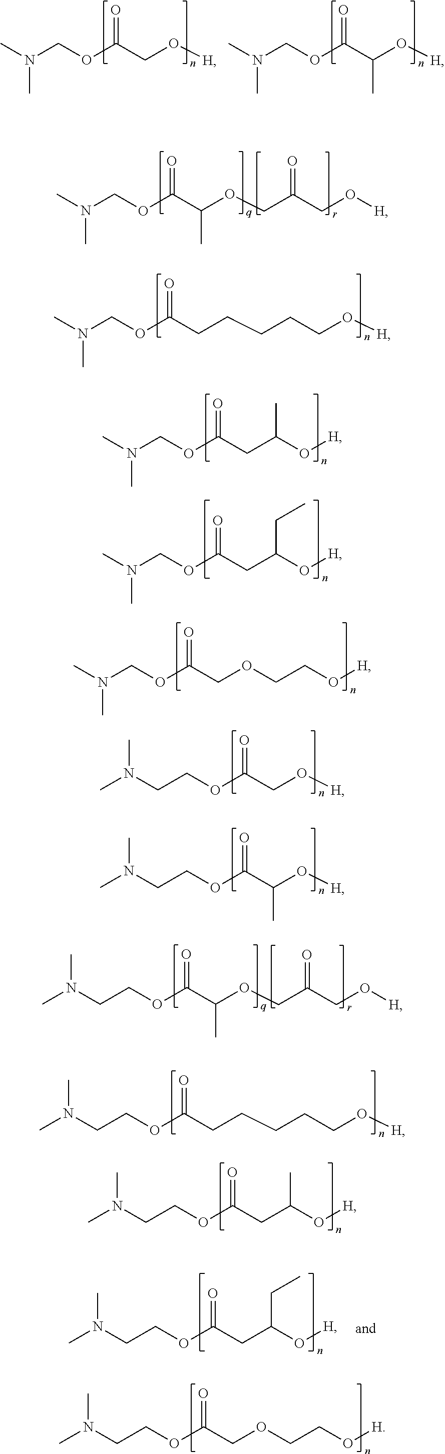

In some examples, the biodegradable polymer is selected from polymers with the following formulae:

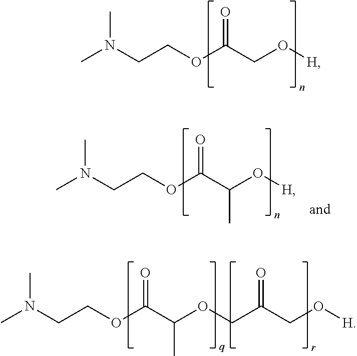

In some examples, the biodegradable polymer may be a polymer with the following formula:

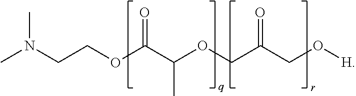

### Microparticles

In another aspect, there is provided microparticles comprising the biodegradable polymer. In some examples, the microparticles comprise any biodegradable polymer defined herein. In some examples, the microparticles consist of or consist essentially of the biodegradable polymer.

In some examples, the microparticles have an average diameter of 2 mm or less, for example, 1 mm or less, 500 μm or less, 100 μm or less, 90 μm or less, 80 μm or less, 70 μm or less, 60 μm or less, 50 μm or less, 40 μm or less, 30 μm or less, 20 μm or less, 10 μm or less, 1 μm or less, 500 nm or less, 100 nm or less, 50 nm or less, 10 nm or less. In some examples, the microparticles have an average diameter of 2 mm or less, for example, 1 mm or less, 500 μm or less, 250 μm or less, 240 μm or less, 230 μm or less, 220 μm or less, 210 μm or less, 200 μm or less, 190 μm or less, 180 μm or less, 170 μm or less, 160 μm or less, 150 μm or less, 100 μm or less, 90 μm or less, 80 μm or less, 70 μm or less, 60 μm or less, 50 μm or less, 40 μm or less, 30 μm or less, 20 μm or less, 10 μm or less, 1 μm or less, 500 nm or less, 100 nm or less, 50 nm or less, 10 nm or less. In some examples, the microparticles have an average diameter of 10 nm or more, for example, 50 nm or more, 100 nm or more, 500 nm or more, 1 μm or more, 10 μm or more, 20 μm or more, 30 μm or more, 40 μm or more, 50 μm or more, 60 μm or more, 70 μm or more, 80 μm or more, 90 μm or more, 100 μm or more, 500 μm or more, 1 mm or more, or 2 mm. In some examples, the microparticles have an average diameter of 10 nm or more, for example, 50 nm or more, 100 nm or more, 150 μm or more, 160 μm or more, 170 μm or more, 180 μm or more, 190 μm or more, 200 μm or more, 210 μm or more, 220 μm or more, 230 μm or more, 240 μm or more, 250 μm or more, 500 nm or more, 1 μm or more, 10 μm or more, 20 μm or more, 30 μm or more, 40 μm or more, 50 μm or more, 60 μm or more, 70 μm or more, 80 μm or more, 90 μm or more, 100 μm or more, 500 μm or more, 1 mm or more, or 2 mm. In some examples, the microparticles have an average diameter of 10 nm to 2 mm, for example, 50 nm to 1 mm, 100 nm to 500 μm, 1 μm to 100 μm, 10 μm to 100 μm, 20 μm to 90 μm, 20 μm to 80 μm, 30 μm to 70 μm, 40 μm to 60 μm, 50 μm to 100 μm. In some examples, the microparticles have an average diameter of 10 nm to 2 mm, for example, 50 nm to 1 mm, 100 nm to 500 μm, 1 μm to 100 μm, 10 μm to 100 μm, 20 μm to 90 μm, 20 μm to 80 μm, 30 μm to 70 μm, 40 μm to 60 μm, 50 μm to 100 μm, 150 μm to 250 μm, 160 μm to 240 μm, 170 μm to 230 μm, 180 μm to 220 μm, 190 μm to 200 μm. In some examples, the average diameter is the mean diameter. In some examples, the mean diameter is calculated from the circular 2D optical surface area. The mean diameter may be the mean diameter calculated from measuring the circular optical surface area of at least 40 particles in each of at least 20 representative images.

### Method of Producing the Biodegradable Polymer

In an aspect, there is provided a method of producing the biodegradable polymer comprising initiating a ring-opening polymerisation of a cyclic ester with an amino alcohol initiator.

The method may comprise combining an amino alcohol with a cyclic ester and performing ring-opening polymerisation to form a biodegradable polymer; wherein the amino alcohol has the formula XR¹OH. In some examples, the cyclic ester may be selected from monoesters having the formula (1) and cyclic diesters having the formula (2), and combinations thereof.

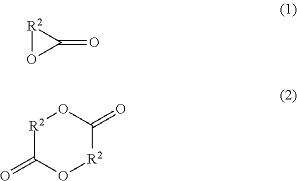

In some examples, X, R¹, and R² are as defined above. In some examples, X is selected from NH₂, NHR³, NR³₂ and NR³₃⁺; R¹ is an alkylene group; each R² is independently selected from a —(CR⁴₂)ₘ— group or a —(CR⁴₂)ₒO(CR⁴₂)ₚ— group; each R³ is independently an alkyl group; each R⁴ is independently selected from hydrogen and an alkyl group; and m, o and p are independently 1 or more.

In some examples, the cyclic ester is any cyclic ester capable of polymerising to form a biodegradable polymer defined herein. In some examples, the cyclic ester is selected from the following compounds,

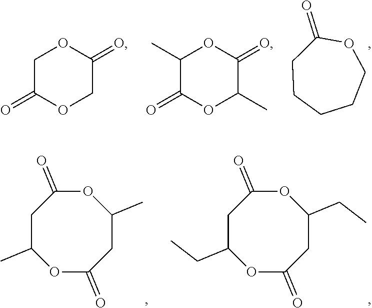

and combinations thereof.

In some examples, the cyclic ester is selected from lactide, glycolide, caprolactone, and combinations thereof. In some examples, the cyclic ester is selected from lactide, glycolide and combinations thereof. In some examples, the cyclic ester is a combination of lactide and glycolide.

In some examples, the cyclic ester is a single cyclic ester or a mixture of cyclic esters. In some examples, the mixture of cyclic esters may be an 85:15 to 50:50 mixture of lactide and glycolide, for example, a 75:25 to 50:50 mixture, or a 1:1 mixture. In some examples, the cyclic ester is a 1:1 mixture of lactide and glycolide.

In some examples, the amino alcohol may be any amino alcohol, for example, any amino alcohol capable of initiating the ring-opening polymerisation reaction with a cyclic ester. In some examples, the amino alcohol may be any amino alcohol capable of forming a biodegradable polymer as defined herein when reacted with a cyclic ester.

In some examples, the amino alcohol may be an amino methanol (for example, (methylamino)methanol or (dimethylamino)methanol), an amino ethanol (for example, (methylamino)ethanol or (dimethylamino)ethanol), or an amino propanol (for example, (methylamino)propanol or (dimethylamino)ethanol). In some examples, the amino alcohol may be (dimethylamino)ethanol.

In some examples, the ring-opening polymerisation is bulk ring-opening polymerisation or solution ring-opening polymerisation. In some examples, the ring-opening polymerisation is bulk ring-opening polymerisation.

In some examples, combining an amino alcohol with a cyclic ester and performing ring-opening polymerisation to form a biodegradable polymer may comprise combining an amino alcohol with a cyclic ester and a catalyst. In some examples, the method may comprise combining an amino alcohol with a cyclic ester, heating and then adding a catalyst to perform the ring-opening polymerisation.

In some examples, performing ring-opening polymerisation may comprise heating. In some examples, the ring-opening polymerisation may be performed at room temperature (e.g., 20° C. to 25° C.). In some example, heating may comprise heating to at least 30° C., for example, at least 40° C., at least 50° C., at least 60° C., at least 70° C., at least 80° C., at least 90° C., at least 100° C., at least 110° C., at least 120° C., at least 130° C., at least 140° C. In some examples, performing ring-opening polymerisation may comprise heating to 200° C. or less, for example, 190° C. or less, 180° C. or less, 170° C. or less, 160° C. or less, 150° C. or less, 140° C. or less, 130° C. or less, 120° C. or less, 100° C. or less, 90° C. or less, 80° C. or less, 70° C. or less, 60° C. or less, 50° C. or less, 40° C. or less, 30° C. or less. In some examples, the ring-opening polymerisation may be performed at a temperature within the range of from 20° C. to 200° C., for example, 25° C. to 190° C., 30° C. to 180° C., 40° C. to 170° C., 50° C. to 160° C., 60° C. to 150° C., 70° C. to 140° C., 80° C. to 200° C., 90° C. to 100° C., 100° C. to 200° C., for example, 110° C. to 190° C., 120° C. to 180° C., 130° C. to 170° C., 100° C. to 160° C., 110° C. to 150° C., or 120° C. to 140° C. In some examples, performing ring-opening polymerisation may comprise heating for at least 12 hours, for example, at least 18 hours, at least 24 hours, at least 36 hours or at least 48 hours. In some examples, performing ring-opening polymerisation may comprise heating for 12 hours to 5 days, for example, 18 hours to 4 days, 24 hours to 3 days, 24 hours to 2 days.

In some examples, the ring-opening polymerisation is performed under an inert atmosphere, for example, a nitrogen atmosphere.

In some examples, the ring-opening polymerisation is catalysed. In some examples, the catalyst may be any catalyst capable of catalysing a ring-opening polymerisation reaction. In some examples, the catalyst may be a metal catalyst, for example, tin(II) 2-ethylhexanoate, 4-dimethylaminopyridine (DMAP), or aluminium isopropoxide.

In some examples, the method may further comprise processing the biodegradable polymer, for example, to form microparticles, fibres, particles or products. In some examples, processing the biodegradable polymer may comprise forming microparticles comprising the biodegradable polymer; electrospinning the biodegradable polymer; extruding the biodegradable polymer; or moulding the biodegradable polymer.

In some examples, the method may further comprise forming particles or microparticles comprising (for example, microparticles of) the biodegradable polymer. In some examples, the particles or microparticles may be formed by an emulsification technique, for example, membrane emulsification, oil-in-water emulsification, water-in-oil emulsification, water-in-oil-in-water emulsification or oil-in-water-in-oil emulsification. In some examples, the emulsion may be formed by homogenisation, sonication, ultrasonication, continuous flow formation, microfluidics, droplet deposition or solvent precipitation. In some examples, the method further comprises forming microparticles comprising the biodegradable polymer by membrane emulsification.

In some examples, membrane emulsification comprises injecting an organic solution comprising the biodegradable polymer into an aqueous solution through a membrane. In some examples, membrane emulsification comprises injecting an organic solution comprising the biodegradable polymer into an aqueous solution of a polymer (such as polyvinyl alcohol) and a salt (such as NaCl) through a membrane, for example, a hydrophilic ring membrane. In some examples, the hydrophilic ring membrane may be a stainless-steel hydrophilic ring membrane. In some examples, the membrane may comprise a pore size of 100 μm or less, for example, 90 μm or less, 80 μm or less, 70 μm or less, 60 μm or less, 50 μm or less, 40 μm or less, 30 μm or less, 20 μm or less or 10 μm or less. In some examples, the membrane may comprise a pore size of 300 μm or less, for example, 250 μm or less, 200 μm or less, 190 μm or less, 180 μm or less, 170 μm or less, 160 μm or less, 150 μm or less, 100 μm or less, for example, 90 μm or less, 80 μm or less, 70 μm or less, 60 μm or less, 50 μm or less, 40 μm or less, 30 μm or less, 20 μm or less or 10 μm or less. In some examples, the membrane may comprise a pore size of 10 μm or more, for example, 20 μm or more, 30 μm or more, 40 μm or more, 50 μm or more, 60 μm or more, 70 μm or more, 80 μm or more, 90 μm or more or 100 μm or more. In some examples, the membrane may comprise a pore size of 10 μm or more, for example, 20 μm or more, 30 μm or more, 40 μm or more, 50 μm or more, 60 μm or more, 70 μm or more, 80 μm or more, 90 μm or more, 100 μm or more, 150 μm or more, 160 μm or more, 170 μm or more, 180 μm or more, 190 μm or more, 200 μm or more, 250 μm or more, or 300 μm or more. In some examples, the membrane may comprise a pore size of 10 μm to 100 μm, for example, 20 μm to 80 μm, 30 μm to 60 μm or 40 μm to 50 μm. In some examples, the membrane may comprise a pore size of 10 μm to 300 μm, for example, 20 μm to 250 μm, 30 μm to 200 μm, 10 μm to 180 μm, 20 μm to 190 μm, 10 μm to 100 μm, for example, 20 μm to 80 μm, 30 μm to 60 μm or 40 μm to 50 μm.

In some examples, the organic solution comprising the biodegradable polymer has a concentration of at least 1% w/v, for example, at least 5% w/v, at least 10% w/v, at least 11% w/v, at least 12% w/v, at least 15% w/v, at least 20% w/v, at least 30% w/v, at least 40% w/v, at least 50% w/v. In some examples, the organic solution of the biodegradable polymer has a concentration of up to 50% w/v, up to 40% w/v, up to 30% w/v, up to 20% w/v, up to 15% w/v, up to 13% w/v, up to 12% w/v, up to 11% w/v, up to 10% w/v, up to 5% w/v, up to 1% w/v. In some examples, the organic solution of the biodegradable polymer has a concentration of 1% w/v to 50% w/v, for example, 5% w/v to 40% w/v, 10% w/v to 30% w/v, 1% w/v to 20% w/v, or 5% w/v to 15% w/v. In some examples, the concentration of the organic solution is dependent on the molecular weight of the biodegradable polymer. In some examples, the organic solution comprises the biodegradable polymer dissolved in an organic solvent, for example, dichloromethane.

In some examples, the organic solution is injected into the aqueous solution at a rate of at least 0.25 mL/min, for example, at least 0.3 mL/min, at least 0.4 mL/min, at least 0.5 mL/min, at least 1 mL/min, at least 2 mL/min, at least 3 mL/min, at least 4 mL/min, at least 5 mL/min. In some examples, the organic solution is injected into the aqueous solution at a rate of up to 5 mL/min, for example, up to 4 mL/min, up to 3 mL/min, up to 2 mL/min, up to 1 mL/min, up to 0.9 mL/min, up to 0.8 mL/min, up to 0.7 mL/min, up to 0.6 mL/min, or up to 0.5 mL/min. In some examples, the organic solution is injected into the aqueous solution at a rate of 0.25 mL/min to 5 mL/min, for example, 0.3 mL to 4 mL/min, 0.4 mL/min to 3 mL/min, 0.5 mL/min to 2 mL/min, 0.6 mL/min to 1 mL/min, 0.3 mL/min to 0.9 mL/min, 0.4 mL/min to 0.8 mL/min, or 0.5 mL/min to 0.6 mL/min.

In some examples, membrane emulsification comprises injecting an organic solution comprising the biodegradable polymer into an aqueous solution through a membrane, collecting the droplets from the emulsion and evaporating the organic phase to form particles, for example, microparticles, comprising the biodegradable polymer. In some examples, the particles are collected by centrifugation. In some examples, the particles are freeze-dried.

In some examples, extruding the biodegradable polymer may comprise three-dimensional printing of the biodegradable polymer. In some examples, the three-dimensional printing may be stereolithography or may comprise photopolymerisation, for example, by digital light processing or UV laser processing.

In some examples, the method may further comprise culturing cells in the presence of the biodegradable polymer. In some examples, the method may further comprise culturing cells in the presence of microparticles comprising the biodegradable polymer.

In some examples, the method further comprises culturing cells in the presence of the biodegradable polymer to form a cell-coated biodegradable polymer. In some examples, the method further comprises culturing cells in the presence of particles comprising the biodegradable polymer to form cell-coated particles comprising the biodegradable polymer. In some examples, the method further comprises culturing cells in the presence of microparticles comprising the biodegradable polymer to form cell-coated microparticles comprising the biodegradable polymer.

### Cell Cultures

In another aspect, there is provided a method of culturing cells comprising combining a biodegradable polymer, particles comprising the biodegradable polymer, or microparticles comprising the biodegradable polymer with a cell culture medium and incubating the composition. In some examples, the method of culturing cells may comprise combining any biodegradable polymer described herein, any particles comprising the biodegradable polymer described herein, or any microparticles described herein with a cell culture medium and incubating the composition.

In some examples, the method of culturing cells may be a method of culturing adherent cells. In some examples, the method of culturing cells may comprise a 2D adherent cell culturing method or a 3D adherent cell culturing method (for example, a suspension culturing method for culturing adherent cells).

In some examples, the method of culturing cells may comprise culturing adherent cells in the presence of the biodegradable polymer, wherein the adherent cells may adhere to the biodegradable polymer during culturing. In some examples, the method of culturing cells may comprise culturing adherent cells in the presence of particles (e.g., microparticles) comprising the biodegradable polymer, wherein the adherent cells may adhere to the particles (e.g., microparticles) comprising the biodegradable polymer.

In some examples, a 2D adherent cell culturing method comprises culturing adherent cells on a 2D flat culturing surface, for example, a film of the biodegradable polymer or a substrate comprising the biodegradable polymer.

In some examples, a 3D adherent cell culturing method comprises culturing adherent cells on a 3D culturing surface, for example, a suspension comprising the biodegradable polymer or a matrix comprising the biodegradable polymer. In some examples, the suspension comprising the biodegradable polymer may comprise particles comprising the biodegradable polymer or microparticles comprising the biodegradable polymer.

In some examples, the particles (e.g., microparticles) comprising the biodegradable polymer may be porous. In some examples, the particles (e.g., microparticles) comprising the biodegradable polymer may have a microporous surface.

In some examples, the method of culturing cells may comprise suspending the biodegradable polymer in a cell culture medium and incubating. In some examples, the method of culturing cells may comprise suspending particles (e.g., microparticles) comprising the biodegradable polymer in a cell culture medium and incubating.

In some examples, the method of culturing cells may comprise suspending the biodegradable polymer in a cell culture medium and incubating under agitation, for example, stirring. In some examples, the method of culturing cells may comprise suspending particles (e.g., microparticles) comprising the biodegradable polymer in a cell culture medium and incubating under agitation, for example, stirring.

In some examples, the method of culturing cells may be a batch culturing method or a perfusion-based culturing method. In some examples, the method of culturing cells may be a surface batch method, a macroporous perfusion method, or a high density macroporous perfusion method. In some examples, the method of culturing cells may be performed in a stirred tank system, a fluidized bed system or packed bed system.

In some examples, the cells grow as a monolayer on the surface of the biodegradable polymer (for example, on the surface of a film of the biodegradable polymer, on the surface of a layer of the biodegradable polymer or a on the surface of a particle (e.g., a microparticle) comprising the biodegradable polymer). In some examples, the cells grow as a monolayer on the surface of particles comprising the biodegradable polymer, for example, microparticles comprising the biodegradable polymer.

The cell culture medium may comprise cells. In some examples, the biodegradable polymer may be combined with the culture medium (i.e., the cell culture medium excluding the cells) before the biodegradable polymer is combined with the cell culture medium.

In some examples, the cells comprise animal cells, for example, anchorage-dependent animal cells. In some examples, the animal cells may be mammalian cells. In some examples, the cells comprise stem cells, β cells, antibody generating cells, chimeric immunoreceptor T cells (CAR T cells), specialised cells or a mixture thereof. In some examples, the stem cells comprise embryonic stem cells, tissue-specific stem cells, mesenchymal stem cells, hematopoietic stem cells, induced pluripotent stem cells or a mixture thereof. In some examples, the specialised cells comprise bone cells, skin cells, muscle cells, cardiac cells, lung cells, intestinal cells or a mixture thereof.

Without wishing to be bound by theory, it is believed that the cells attach to the biodegradable polymer through the X group, which may be cationic in the cell culture medium.

In some examples, the method of culturing cells may comprise combining the biodegradable polymer or the particles (e.g., microparticles) comprising the biodegradable polymer with a cell culture medium and incubating under agitation, for example, agitation by stirring. In some examples, the method of culturing cells may comprise combining the biodegradable polymer or the particles (e.g., microparticles) comprising the biodegradable polymer with a cell culture medium and incubating under static conditions before incubating under agitation, for example, agitation by stirring. In some examples, the method of culturing cells may comprise combining the biodegradable polymer or the particles (e.g., microparticles) comprising the biodegradable polymer with a cell culture medium and incubating under static conditions.

In some examples, the biodegradable polymer, particles comprising the biodegradable polymer or the microparticles comprising the biodegradable polymer were sterilised (for example, by UV irradiation) before being combined with the culture medium or the cell culture medium.

In some examples, the composition is incubated until at least 1×10³ cells per mg of biodegradable polymer is formed, for example, at least 1.5×10³ cells per mg of biodegradable polymer, at least 2×10³ cells per mg of biodegradable polymer is formed. In some examples, the composition is incubated until at least 1×10³ cells per mg of particles (e.g., microparticles) comprising biodegradable polymer is formed, for example, at least 1.5×10³ cells per mg of particles (e.g., microparticles) comprising biodegradable polymer, at least 2×10³ cells per mg of particles (e.g., microparticles) comprising the biodegradable polymer is formed.

In some examples, the method of culturing cells may further comprise detaching the cells from the biodegradable polymer, for example, from the particles (e.g., microparticles) comprising the biodegradable polymer. In some examples, the method of culturing cells may not comprise detaching the cells from the biodegradable polymer, for example, from the particles (e.g., microparticles) comprising the biodegradable polymer. For example, the adhesion of the cells to the biodegradable polymer may not affect the utility of the cells.

In some examples, the cells may be cultured for the production of vaccines, enzymes, hormones, antibodies, interferons, nucleic acids. In some examples, the cells may be cultured for implantation of the cells themselves. In some examples, the cells and the biodegradable polymer may be implanted together, that is, there may be no need to harvest the cells after cell culturing is completed.

### Compositions and Products

In another aspect, there is provided a composition comprising cells supported on the biodegradable polymer or on the particles (e.g., microparticles). In some examples, there is provided a composition comprising cells supported on any biodegradable polymer described herein or on any particles (e.g., microparticles) comprising a biodegradable polymer described herein.

In some examples, the composition may be a supported tissue a supported tissue structure, or a tissue scaffold. In some examples, the cells may be any cells, for example, any cells described herein. In some examples, the cells may comprise stem cells (e.g., embryonic stem cells, tissue-specific stem cells, mesenchymal stem cells, hematopoietic stem cells, or induced pluripotent stem cells), β cells, antibody generating cells, or chimeric immunoreceptor T cells (CAR T cells), specialised cells (e.g., bone cells, skin cells, muscle cells, cardiac cells, lung cells, intestinal cells) or a mixture thereof.

In some examples, there no need to harvest the cells from the biodegradable polymer. In some examples, the composition can be injected or implanted into a human or animal body. In some examples, the composition comprising cells and the biodegradable polymer can be used in cell culturing, cell scale up and cell delivery without requiring a cell harvesting step after cell expansion. In some examples, the composition can be directly injected or implanted.

In another aspect, there is provided a tissue scaffold, a microcarrier for biologics or a medical implant comprising the biodegradable polymer or the microparticles. In some examples, the microcarrier for biologics may be a microcarrier for genetic material, drugs, cells or antibodies. In some examples, the microcarrier for biologics may comprise or be the biodegradable polymer described herein. In some examples, the microcarrier for biologics may comprise or be the microparticles comprising the biodegradable polymer described herein.

In some examples, the particles (e.g., microparticles) comprising the biodegradable polymer comprise a microcarrier for biologics. In some examples, the microcarrier for biologics may comprise a microcarrier for genetic material, drugs, cells or antibiotics.

The biodegradable polymer described herein may be used in tissue engineering or regenerative medicine. The microparticles comprising the biodegradable polymer described herein may be used in tissue engineering or regenerative medicine. In some examples, the biodegradable polymer or the microparticles comprising the biodegradable polymer may be for use in tissue engineering or regenerative medicine. In some examples, the biodegradable polymer or the microparticles comprising the biodegradable polymer may be for use as a medicament.

### EXAMPLES

The following illustrates examples of the methods and other aspects described herein. Thus, these Examples should not be considered as limitations of the present disclosure, but are merely in place to teach how to make examples of the present disclosure.

### Materials

Reagents were used as purchased, unless stated otherwise. Dimethylaminoethanol (DMAEtOH; ≥99.5%); citric acid (≥99.5); polyvinyl alcohol (PVA; Mw 13,000-23,000, 87-89% hydrolyzed); sodium chloride (NaCl; ≥99.5%); Hoechst 33258 fluorescent dye; paraformaldehyde were purchased from Sigma-Aldrich (UK). D,L-Lactide (>98.0%), glycolide (>98.0%) and tin(II) 2-ethylhexanoate (>85.0%) were purchased from Tokyo chemical industry. Tetrahydrofuran (THF; ≥99.9%); dichloromethane (DCM; ≥99.5%); acetone (≥99.8%), methanol (≥99.8%); 2-propanol (≥99.7%) were purchased from VWR International. 1-Propanol (anhydrous, 99.9%) was purchased from Alfa Aesar™. Deuterated chloroform (CDCl₃; ≥99.8%) was purchased from Acros Organics. Sodium hydroxide (NaOH; ≥99.0%) was purchased from Merck Millipore. Adipose tissue-derived stem cells (ADSCs), ADSCs basal media and supplements fetal bovine serum (FBS; 10%), L-glutamine (1%), gentamicin-amphotericin (0.1%); trypsin; trypsin neutralizing solution were purchased from Lonza. Phosphate buffered saline (PBS) tablets; trypan blue were purchased from Thermo Fisher Scientific. CellTiter 96® AQueous One Solution Cell Proliferation (MTS) Assay was purchased from Promega. CyQUANT™ LDH Cytotoxicity Assay; LIVE/DEAD™ Viability/Cytotoxicity Kit, for mammalian cells were purchase from Thermo Fisher Scientific.

### Methods

### Synthesis of Unmodified PLGA and PLGA_{DMAE} Polymers

The PLGA_{DMAE} polymer was synthesized via the bulk ring-opening polymerization (ROP) technique. First, a round-bottom flask containing the monomers D,L-lactide (11.3 g, 78.5 mmol) and glycolide (9.1 g, 78.5 mmol), and the initiator 2-dimethylaminoethanol (0.1 g, 1.1 mmol, 117 μL) was degassed with nitrogen gas for 1 h and the flask was tightly sealed with a septum. The mixture was then gradually heated to 140° C. under stirring and a nitrogen atmosphere. Once the mixture was completely dissolved, the catalyst tin(II) 2-ethylhexanoate (0.046 g, 0.1 mmol) was added via a needle. The mixture was left to stir at a speed of 250 rpm and 140° C. for a further 48 h. After this time, the reaction mixture was then cooled and the solidified polymer was collected from the flask.

To directly compare the PLGA_{DMAE} to the unmodified PLGA, the latter was synthesized under the same conditions except the DMAE was replaced with anhydrous 1-propanol as the ROP initiator. Samples of the PLGA and PLGA_{DMAE} polymers were collected for subsequent analyses.

### Fabrication of PLGA and PLGA_{DMAE} Microparticles (MPs)

The PLGA and PLGA_{DMAE} MPs were synthesized via the membrane emulsification technique, by using the Micropore LDC-1 dispersion kit (Micropore technologies, UK). A continuous stabilizing solution was prepared by mixing PVA (20 g) and NaCl (26 g) in ultrapure water (1.8 L) under stirring and at 40° C. overnight until complete dissolution has occurred. The solution was then vacuum filtered using a Buchner funnel. To this solution, 46 g of DCM solvent was gradually added and stirred for 1 h. The PLGA and PLGA_{DMAE} (12.5% w/v) polymers were dissolved separately in DCM (14 mL) to produce the organic phase. To generate emulsion droplets, the organic phase was injected via a stainless-steel hydrophilic ring membrane (40 μm pore size) into the continuous phase (130 mL) at a rate of 0.50 mL/min, and a pre-adjusted stirring speed of 600 rpm. The droplets formed were collected and transferred into a beaker. The mixture was then left to stir overnight under a fume hood to evaporate the organic phase, resulting in the formation of solidified MPs. The MPs were then collected, transferred into a Buchner funnel and washed with ultrapure water several times to remove the residual PVA polymers. Finally, the MPs were collected via centrifugation at 1000 rpm for four minutes. The final MPs were freeze-dried to obtain a loose, dry powder and stored under cold (2° C. to 8° C.), dry conditions until further use.

### Characterisation

### Molecular Weight

The molecular weight of PLGA and PLGA_{DMAE} were calculated by using Gel Permeation Chromatography (GPC) analysis. The PL-GPC 50 system was used in conjunction with a refractive index detector (a deflection-type detector, λ=890 nm, cell volume=6 μL, pressure rating 50 kPa, wetted materials (316 SST, quartz)). A guard column (PLgel 5 μm MiniMIX-C, 50 mm×4.6 mm) was connected with two columns (PLgel 5 μm Mixed-D, 300 mm×7.5 mm) and maintained at 30° C. The GPC machine was calibrated by using Agilent EasiVial GPC poly(methyl methacrylate) calibration standards (PMMA; molar mass range: 3×10⁵-6×10⁵ Da). To perform the analysis, a sample of PLGA and PLGA_{DMAE} (approx. 1 mg/ml) was dissolved in THF (HPLC grade) and injected at a flow rate of 1 mL/min. The data were collected and analysed using CIRRUS™ data stream software (version 1.1).

### NMR Spectroscopy

¹H NMR spectroscopy was used to confirm the polymer structures, using the Bruker 400 MHz spectrometer. For this, a small quantity of the PLGA and PLGA_{DMAE} polymers was dissolved separately in deuterated chloroform (CDCL₃; 1.2 mL) and ¹H NMR spectra with an average of 16 scans were recorded for each sample. The data was analysed using TopSpin™ NMR software v. 4 and processed with MestReNova software v. 6.0.2-5475.

### Thermogravimetric Analysis

Thermogravimetric analysis (TGA) was performed on the PLGA and PLGA_{DMAE} polymers. Approximately 1-5 mg of the polymers was placed in tared unsealed standard aluminium pans (TA instruments). The data was recorded under a nitrogen atmosphere and analysed using a TGA 5500 analyser (TA Instruments). The samples were heated from room temperature to 400° C. at heating a rate of 10° C./min. Two sets of data were collected including a weight loss (%) profile and a derivative weight (%/° C.) as a function of temperature. All measurements were recorded by using TRIOS software (version 5.2).

### Glass Transition Temperature (T_{g})

Differential Scanning Calorimetry (DSC) analysis was performed on the polymers to determine the glass transition temperature (T_{g}). Approximately 1-5 mg of the polymers was placed in sealed standard aluminium pans (TA instruments), and an empty sealed standard aluminium pan (TA instruments) was used as a reference. The data was collected using a DSC 2500 analyser (TA Instruments) and a under nitrogen atmosphere. For this, the samples were equilibrated first at −10° C., and then heated to 150° C. at a rate of 10° C./min, then cooled to −10° C. at a rate of 10° C./min. Finally, the samples were heated again to 150° C. at the same heating rate of 10° C./min (to obtain three cycles in total). The T_{g} of the polymers was obtained from the second heating event to remove any interference from the residual moisture in the samples. A heat flow measurement (W/g) as a function of increasing temperature profile was then obtained. All measurements were recorded using TRIOS software (version 5.2).

### Microparticle Size and Size Distribution Analysis

The mean particle size and particle size distributions for PLGA and PLGA_{DMAE} MPs were measured using an optical EVOS XL Core phase contrast microscope (Thermo Fisher Scientific). To calculate the span, the average diameter (D) in μm of the solidified microparticles was calculated from the circular 2D optical surface area by using the oval tool from ImageJ software (version 1.51). This tool was used to select the circumference of the particles. 20 representative images of PLGA and PLGA_{DMAE} MPs were taken at 20× magnification, and a minimum of 40 particles per image were used for data analysis. The span was then calculated from the D10, D50 and D90 corresponding to the diameters at the 10^{th}, 50^{th} and 90^{th} percentiles, respectively. The D90 was subtracted from the D10 values and the result was then divided by the D50. Distribution histograms were created with SPSS statistical software (IBM Corp. Released 2020. IBM SPSS Statistics for Windows, Version 27.0. Armonk, NY: IBM Corp) to illustrate the dispersity of MPs.

The morphology of the MPs was also analysed by Scanning Electron Microscopy (SEM) imaging. Briefly, freeze-dried MPs were loaded onto the aluminium stubs with carbon tabs and then gold coated with a Quorum Technologies Polaron Gold Sputter Coater (rotary stage, 30 sec, 20 mA, 2.2 kV, average distance from Au target: 55 mm). The MPs were then imaged by using a Zeiss Gemini SEM 300 at an accelerating voltage of 2 kV.

To investigate if the UV sterilization process had any impact on structure and morphology of the MPs, confirmatory (¹H NMR) and SEM analyses were performed on the MPs before and after the UV sterilization process. The samples were analysed following the protocols described earlier for each technique. To further supplement the analysis, Fourier-Transform Infrared Spectroscopy (FT-IR) was conducted on the PLGA and PLGA_{DMAE} MPs before and after UV irradiation. FT-IR spectra were recorded on a Bruker Vertex 70V FTIR Spectrometer equipped with a Golden Gate™ Heat-enabled ATR accessory (Specac LTD, UK) in absorption mode against wavenumber. The OPUS software was used to record the spectra at 4 cm⁻¹ resolution using 32 scans. The wavenumber range of the spectra was selected from 550 to 4000 cm⁻¹.

### Cell Culture

Human adipose-derived Stem cells (ADSCs; PT-5006, Lot number 0000439846) were purchased from Lonza (statement from the supplier confirms the cells were obtained under ethical conditions with donor consent and a tissue acquisition letter is available upon request). Cells were received in passage one and expanded to passage three by using monolayer culture conditions, following the manufacturer protocols (Catalogue number PT-4505; Lonza). Cells were cultured in ADSCs Basal Media (supplemented with 10% FBS, 1% L-glutamine, and 0.1% gentamicin amphotericin) and maintained in a humidified atmosphere with 5% CO₂ in the air at 37° C. prior to the experiments.

### Cell Attachment

The freeze-dried PLGA and PLGA_{DMAE} MPs were sterilised by standard UV irradiation (for 1.5 h, λ=254-260 nm). The MP powder was loaded into a glass vial and rotated on a laboratory roller to maximise exposure to UV irradiation. The sterilised MPs were prepared in cultured conditions. Briefly, 10 mg of the freeze-dried MPs were suspended in 1 mL of the complete cell culture media for 12 h at room temperature. Following this, the media was removed from the MP suspension via centrifugation at 1000 rpm for four minutes followed by gentle aspiration of the supernatant, leaving the conditioned MPs in place.

Two methods were followed to study the cell attachment to the MPs. In Method 1, 20,000 cells suspended in 250 μL complete media were mixed with 10 mg of the MPs to obtain a final concentration of 2×10³ cells·mg⁻¹ of MPs (the calculated surface area of the MPs equals 0.4 cm²·mg⁻¹). This mixture was directly transferred into the well of a 24-well cell culture plate and topped up to 1 ml with cell culture media.

In Method 2, an in-house developed protocol to maximise cell attachment was used that mixes the MPs and cells in closer proximity. Cells and MPs were mixed by using the same concentrations as for Method 1. From this mixture, 20 μL of the cell and MP mixture was pipetted (with wide orifice pipette tips (size 20-200 for P200 pipette)) onto an inverted lid of a 60 mm diameter petri dish, which was gently flipped over and placed on top of the petri dish containing 5 mL of PBS in order to maintain a humidified environment. After 24 h of incubation and under the conditions described for Method 1, all the droplets collected from each Petri dish lid were transferred into one well of a 24-well tissue culture plate, which had been filled with 500 μl of cell culture media. After the transfer of the droplets, each well was topped up to 1 mL with complete media. The mixture of cells and MPs was maintained in a humidified atmosphere with 5% CO₂ at 37° C. for predetermined time intervals (1, 5, 7, 14 days; or 7, 14, 21 days for the degradation study). All experiments were conducted under static conditions and the cell culture media was refreshed every three days (approximately, 600 μL of cell culture media was gently removed and replaced by fresh media).

### Cytotoxicity Study

ADSCs were cultured on PLGA and PLGA_{DMAE} MPs, as previously described (method 2), in 24-well cell culture plates for 1, 5, 7, and 14 days, and an LDH analysis was performed by following the manufacturer's instructions. Briefly, 50 μl of cell culture medium was withdrawn from tissue culture plates and mixed with the CyQUANT™ LDH Cytotoxicity Assay Kit LDH reagent in a 1:1 ratio. After 30 minutes of incubation at room temperature and storage in a dark place, the LDH stop solution was added. The absorbance values were measured at 490 and 680 nm with a microplate reader CLARIOSTAR; BMG Labtech). Background absorbance at 680 nm was subtracted from the absorbance at 490 nm. Cell culture media without cells was used as a blank control.

Phase contrast microscopy was used to monitor all cell culture conditions. Representative images were taken 1, 5, 7 and 14 days after culture began by using an EVOS XL Core (Thermo Fisher Scientific) phase contrast microscope. Images were taken with 10× or 20× magnification and processed by using ImageJ software (version 1.51).

### Live/Dead Assay

ADSCs were cultured on PLGA and PLGA_{DMAE} MPs in 24-well cell culture plates (method 2) for 1, 7, and 14 days. Approximately, 600 μL of media was removed from the culture dishes and MPs were washed once with 600 μL of PBS. Then, the PBS was gently removed. Freshly prepared Calcein AM and Ethidium homodimer-1 solution (2 and 4 μM, respectively) was prepared following the manufacturer's instructions, added to the cell and MP mixture, and incubated for 40 min in the dark at room temperature. Next, imaging was performed by using a Zeiss Axiovert 200M microscope using Axiovision software. Images were taken with a 20× magnification Plan-Neofluar (NA 0.5) objective and a Zeiss AxioCam HRm CCD camera. Fluorescence was excited either at 494-517 nm (Calcein) or 528-617 nm (Ethidium homodimer-1). Merged images of Calcein (green), Ethidium homodimer-1 (red) and Bright Field were created in Image J software (version 1.51). Live and dead cells were counted in every picture, using a minimum of three random locations per sample.

### Cell Attachment Analysis

SEM imaging was used to investigate cell attachment onto the PLGA and PLGA_{DMAE} MPs. The ADSCs were cultured on PLGA and PLGA_{DMAE} MPs, as previously described in 24-well cell culture plates (method 2) for 7 days (this was considered a midpoint between start of the culture and sufficient cell attachment and growth). Approximately, 600 μL of media was removed, and the remaining cell and MP mixture was fixed with freshly prepared 4% paraformaldehyde in PBS for 30 min at room temperature. Fixed cell and MP mixture was washed three times with PBS and centrifuged at 1000 rpm for four minutes, and then cells and MPs were collected and freeze-dried. The freeze-dried powder was loaded onto aluminium stubs with carbon tabs. The samples were gold coated with a Quorum Technologies Polaron Gold Sputter Coater (rotary stage, 30 sec, 20 mA, 2.2 kV, average distance from Au target: 55 mm) and subsequently imaged by using a JSM 5900LV (JEOL) SEM at an accelerating voltage of ˜10 kV.

### Degradation Study

PLGA and PLGA_{DMAE} MPs were prepared as previously described. The MPs were immersed in 2 mL of PBS, incubated at 37° C. in a water bath for 7, 14, and 21 days, and samples were then collected and freeze-dried.

First, macroscopic and morphological changes to the particles were studied by using SEM (samples were prepared following the same protocol as described above). SEM images of the MPs were acquired by using a Zeiss Gemini SEM 300 at an accelerating voltage of 2 kV.

Second, the molecular degradation of the MPs was examined by using GPC. The freeze-dried MPs were dissolved in 2 mL of THF (HPLC grade) for 24 h at room temperature. After complete dissolution, the samples were injected and analysed by using the previously described protocol. The shift in the number-average molecular weight (Mn) of the PLGA and PLGA_{DMAE} MPs was analysed and compared to that of un-degraded polymers.

### Statistical Analysis

For the particle size and particle size distribution, frequency distribution histograms were created with SPSS statistical software to illustrate the dispersity in microparticle diameter with associated Span values. A bar chart was plotted to compare the mean particle size between PLGA and PLGA_{DMAE} MPs representing the mean particle size (μm)±SD (n=20 representative images of PLGA and PLGA_{DMAE} MPs). A two-tailed unpaired t test was used to assess significant differences between the particle size of both groups (ns: non-significant).

In regard to the cytotoxicity study by using the LDH assay, the data are presented as optical density values (OD)±SD (n=3 independent cell culture replicates). Differences between groups were assessed via a two-way ANOVA with a Tukey's multiple comparison test (Ns: non-significant, *p=0.03, ***p<0.0002, ****p<0.0001).

In the Live/Dead staining experiment, data are presented as the average of counted cells per field±SD (n=3 independent technical cell culture replicates) in three random locations, and thus using a total of 9 images per sample (Ns: non-significant).

### Results

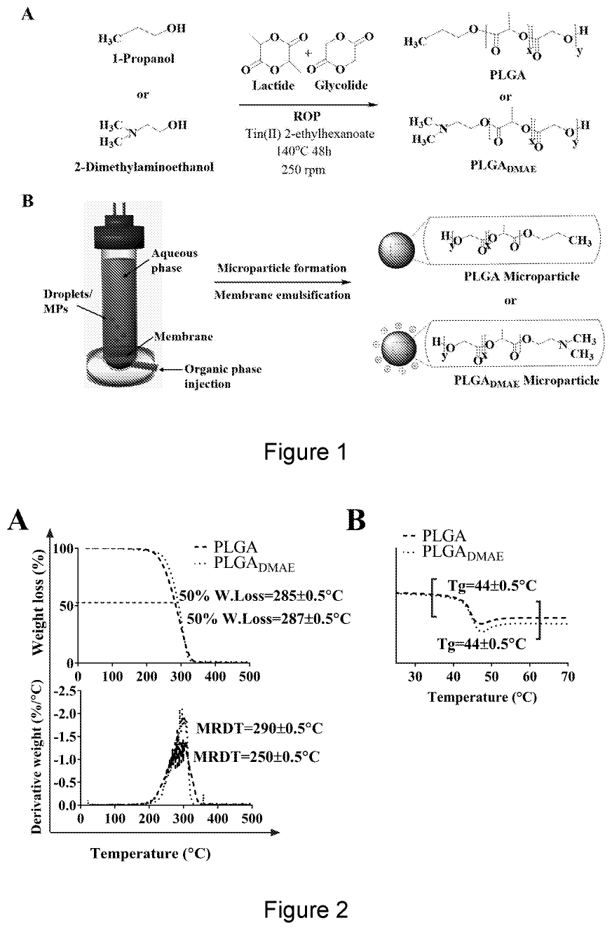

FIG. 1A is a schematic illustration of the one-step synthesis of the 2-dimethylaminoethanol-terminated poly(lactic-co-glycolic acid) polymer (Example polymer; PLGA_{DMAE}) and the propanol-terminated poly(lactic-co-glycolic acid) polymer (Comparative polymer; PLGA). FIG. 1B schematically illustrates the process for fabricating the microparticles.

The theoretical feed ratio and ROP polymerization conditions for both PLGA and PLGA_{DMAE} polymers are summarized in Table 1 (below). The GPC analysis showed that the number-average molecular weight (Mₙ) and polydispersity (PDI) of both polymers (PLGA and PLGA_{DMAE}) were comparable as measured against the PMMA standards.

TABLE 1
PLGA and PLGA_{DMAE} synthesis feed ratio (mol %), relative
molecular weight (g · mol⁻¹), polydispersity and
yield (%) as measured by GPC analysis and using PMMA as standards.

| | Theoretical Feed | | | Relative molecular | | | |
|---|---|---|---|---|---|---|---|
| | ratio [mol %] | | | weight [g/mol] | | | Yield |
| Polymer | Lactide | Glycolide | Initiator | Mₙ | M_{w} | Dispersity | [%] |
| PLGA | 50 | 50 | 1 | 26,563 | 42,980 | 1.6 | >90 |
| PLGA_{DMAE} | 50 | 50 | 1 | 26,400 | 41,381 | 1.6 | >90 |

The chemical structures of the PLGA and PLGA_{DMAE} polymers were confirmed by ¹H NMR analysis. The spectra for both polymers confirmed the main structure and peaks for the repeating monomer units of lactic acid and glycolic acid. The composition was calculated to confirm the 50/50 ratio by integrating the ¹H NMR peaks of the two repeating units. The ¹H NMR spectra of both polymers showed the typical peaks characteristics for each monomer with chemical shifts detected at 5.2 ppm (CH, 1H) and 1.6 ppm (CH₃, 3H) for lactic acid, and at 4.8 ppm (CH₂, 2H) for glycolic acid.

The thermal properties of the polymers were analysed by using both thermogravimetric analysis (TGA) and differential scanning calorimetry (DSC) to investigate whether the insertion of the new DMAE terminal functional group affects the physicochemical properties of the new pre-functionalized PLGA_{DMAE} polymer. As can be seen in FIG. 2A, there was a 50% weight reduction at 287° C. and 285° C. for PLGA and PLGA_{DMAE}, respectively. The derivative weight supported these findings, demonstrating that the maximum rate of degradation temperature (MRDT) of the two polymers were also similar.

A similar observation was also recorded from the DSC analysis which was used to determine the glass transition temperature (T_{g}) of the two polymers (FIG. 2B). The results show that both polymers possess a similar T_{g} at 44° C., this value is broadly similar to the previously reported T_{g} for PLGA polymers having 50:50 ratio of composition of lactic acid to glycolic acid units. These analyses confirmed that the thermal profiles were comparable, showing negligible differences between the two polymers.

In the next step, MPs were fabricated via the single oil-in-water membrane emulsification technique (FIG. 1B). A diameter of the MPs of equal to or greater than 100 μm was targeted since this size range is relatively close to that of commercially available MPs that are used as a cell culture substrate in suspension cell culture systems.

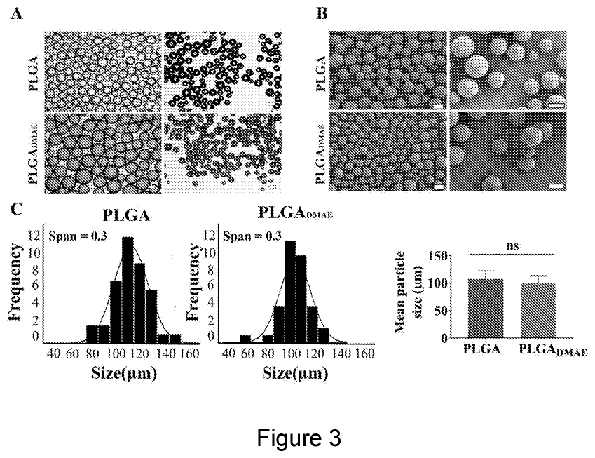

The PLGA and PLGA_{DMAE} MPs were both prepared under the same emulsification parameters and polymer concentrations. Preliminary work (data not shown) suggested that a polymer concentration of 12.5% w/v (adjustable based on the polymer molecular weight) was sufficient to achieve MPs within the target size range. Under the described conditions (see the method described above), MPs were successfully fabricated from both polymers. The comparative size and morphology of fresh droplets, solidified MPs, and freeze-dried MPs derived from the PLGA and PLGA_{DMAE} polymers were assessed using phase contrast microscopy (FIG. 3A) and SEM imaging (FIG. 3B). The phase contrast and SEM imaging confirmed the successful formation of spherical MP morphologies. Further analysis of the phase contrast images demonstrated that the distribution of the particle sizes was unimodal and possessed little variance (FIG. 3C), suggesting a near-uniform particle sizes distribution for both PLGA and PLGA_{DMAE} MPs. The calculated Span values from the phase contrast imaging were also compared between the PLGA and PLGA_{DMAE} MPs and showed a value of 0.3 suggesting a narrow particle size distribution. Furthermore, the mean particle size and relative standard deviation were also plotted as bar charts (FIG. 3C), which indicate that there was no significant difference between the two MPs groups. These data suggest that the pre-functionalization of the polymer did not alter the size or morphology of the MPs.

With the uniformity of the MP size and morphology between the two polymers established, the effect of the surface functionalization on the cytotoxicity profile of the microparticles was assessed. Prior to the cytotoxicity assays, the morphology and chemical properties of the PLGA and PLGA_{DMAE} microparticles were investigated before and after UV sterilization by using SEM imaging, FT-IR and ¹H NMR spectroscopy. The results confirmed that no changes occurred as a result of the sterilization process.

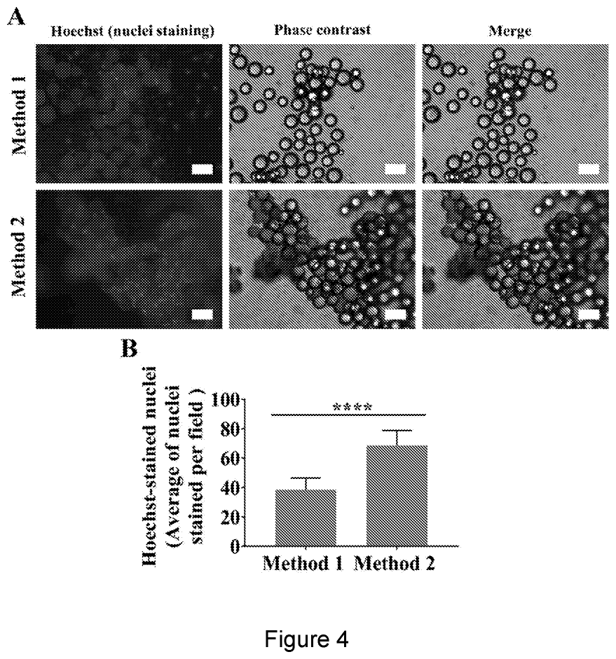

Two methods were investigated to evaluate the efficiency of seeding ADSCs onto the MPs. Method 1 followed a standard protocol involving mixing cells with the MPs in the 24-well plate tissue culture plastic, whereas in Method 2 the cells were mixed with MPs in the form of hanging droplets for 24 h to enhance the exposure of the cells to the MPs in closer proximity. As can be seen in FIG. 4, the nuclei staining and phase contrast imaging suggested that the aggregation of the ADSCs with the MPs was significantly higher in Method 2 compared to that of Method 1. Therefore, method 2 has been used for the rest of the work.

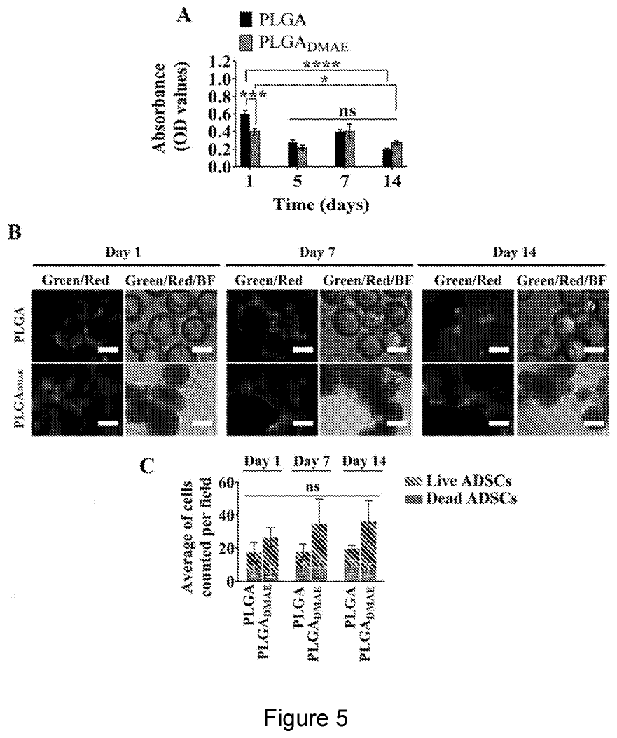

A cytotoxicity study on the MPs co-cultured with ADSCs by using LDH and LIVE/DEAD assays was performed with the results analyzed at days 1, 5, 7 and 14 post-culture. The absorbance value of Formazan (directly proportional to the amount of LDH released into the medium) was recorded as a function of time post culture. In FIG. 5A, a significantly lower concentration of LDH was shown to be released into the medium of the PLGA_{DMAE} MPs group on day 1 post-culture compared with that for the PLGA MPs, which suggests a higher survival rate. At this stage, the difference in the observed LDH concentration between the two groups may be due to the differential attachment of the ADSCs to the PLGA and PLGA_{DMAE} MPs within the first 24 h. However, the differences in LDH concentrations observed between the two groups on day 5, 7 and 14 post-culture were not statistically significant, which may be because the remaining cells were either adhered to the surface of the PLGA_{DMAE} MPs or to the surface of the tissue culture plastic when co-cultured with the unmodified PLGA MPs. This is supported by the fact that the phase contrast imaging shows that the ADSCs were attached to the PLGA_{DMAE} MPs, whereas for the unmodified PLGA MPs group, cells were mostly adhered to the tissue culture plastic.

Taken together, these data suggest that following the successful attachment and survival advantage in the first 24 h period, the PLGA_{DMAE} MPs maintained the already desirable low toxicity profile of PLGA polymers. Furthermore, the LIVE/DEAD viability assay indicated that there was no significant difference in the viability of the cells co-cultured with either PLGA_{DMAE} or PLGA MPs over the cultured time period (FIG. 5B-C).

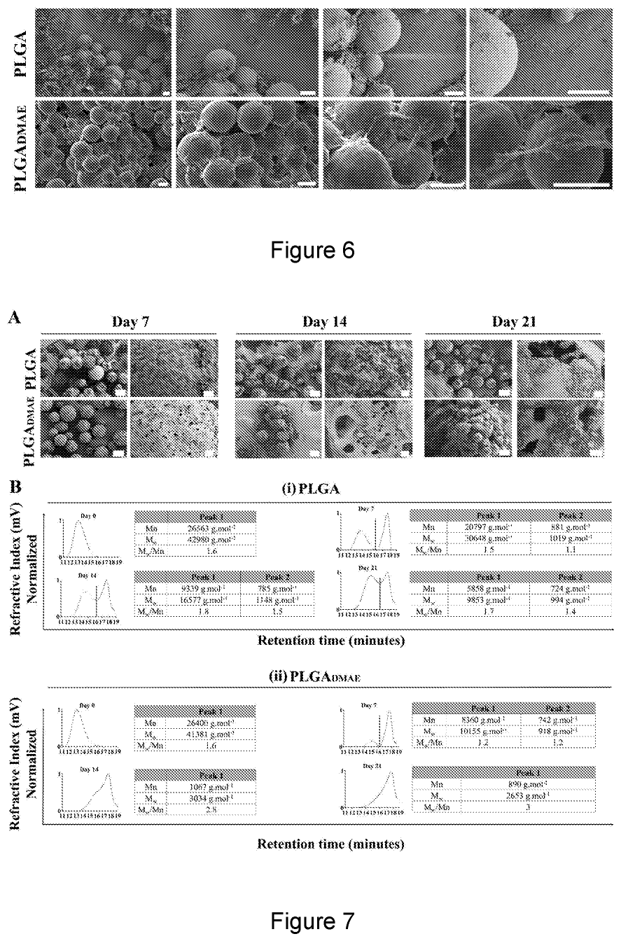

Although the previous assays show that the PLGA_{DMAE} MPs were well tolerated when co-cultured with the ADSCs, more investigations were required to show that cells can attach to the surface of the PLGA_{DMAE} MPs under physiological conditions. Thus, ADSCs were co-cultured with PLGA and PLGA_{DMAE} MPs for 7 days and surface cell attachments were investigated by using SEM imaging (FIG. 6). A pilot study (data not shown) demonstrated that day 7 was the most appropriate time point to image the attachment of the ADSCs to the MPs as it represents a midpoint between early cell attachment and over-confluent cell culture conditions. It was strongly evident from the analysis that the PLGA_{DMAE} MPs readily attach to the co-cultured ADSCs (FIG. 6, bottom row), and that no or poor attachment of the ADSCs to the PLGA MPs is seen (FIG. 6, top row). Once again, the SEM imaging suggested that, when using PLGA MPs, the ADSCs were predominantly attached to the tissue culture plastic rather than to the MPs (FIG. 6, bottom row).

In addition, the ADSCs attached to the PLGA_{DMAE} MPs had different morphologies compared with those co-cultured with the PLGA MPs. Cells attached to the PLGA_{DMAE} MPs adopted elongated morphologies, gripping and spreading across the different MPs surfaces, which suggests the presence of a strong interaction at the interface between the MPs and the cells (FIG. 6, bottom row). In contrast, cells co-cultured with the unmodified PLGA MPs were attached to the tissue culture plastic and displayed typical 2D monolayer flat morphologies (FIG. 6, top row).

Therefore, without wishing to be bound by theory, it is thought that the cell attachment to the surface of the PLGA_{DMAE} MPs may be due to the presence of a weak electrostatic interaction between the positively charged (at physiological pH) DMAE functional groups available on the surface of the PLGA_{DMAE} MPs and the polyanionic cell membranes. The lack of any cell adhesion moieties on the unmodified PLGA MPs resulted in poor or no cell adhesion. Thus, the PLGA_{DMAE} MPs readily promote the attachment of the clinically relevant ADSCs at physiological condition.

Next, to evaluate the degradation profile and morphology of the PLGA and PLGA_{DMAE} MPs, the MPs were re-suspended in PBS (pH 7.4 and at 37° C.) and samples were taken for analysis at days 7, 14, and 21 post-incubation. First, SEM imaging was used to observe the changes in the morphology of the MPs, namely changes that occur due to surface and bulk erosion. To demonstrate these changes to the surface and bulk of the MPs, SEM images were taken at low and high magnifications, respectively. In addition, GPC profiles were also monitored to characterize the changes to the molecular weight and molecular weight distributions. As shown in FIG. 7A, on day 7 of incubation, the morphologies of the PLGA and PLGA_{DMAE} MPs were broadly similar. However, SEM images taken on days 14 and 21 showed the appearance of deformation and coalescence in the structures of PLGA_{DMAE} MPs. Concomitant with this was the presence of larger pore sizes across the surfaces and bulk of the MPs, indicating ongoing surface degradation. In contrast, the PLGA MPs preserved their spherical morphologies on days 14 and 21 but showed early signs of surface erosion, indicated by the appearance of surface cracking. However, no sign of bulk erosion of the PLGA MPs was observed.

The recorded GPC profiles for the samples analysed from the cultured PLGA MPs, (FIG. 7Bi), showed a shift in the molecular weight from a unimodal (at day 0) to a bimodal (at day 7, 14 and 21) distribution, suggesting a slow and partial reduction in the molecular weight from a higher to a lower range. However, the GPC profile for the PLGA_{DMAE} MPs showed faster and more uniform degradation as depicted by the persistence of a unimodal distribution throughout the study (FIG. 7Bii). This was also supported by the calculated number average molecular weight values, which showed a faster reduction in the molecular weight of the PLGA_{DMAE} compared with that of the PLGA MPs.

Without wishing to be bound by theory, it is believed that the insertion of the hydrophilic DMAE groups into the terminal structure of the PLGA polymers may be responsible for enhanced molecular degradation of the PLGA_{DMAE} polymer compared to that of the unmodified PLGA. Similarly, the morphological changes observed with the PLGA_{DMAE} MPs may be due to enhanced water diffusion and thus increased accessibility of water molecules to weak and hydrolytically labile ester bonds. Nonetheless, the overall benefit of faster degradation of the PLGA_{DMAE} MPs compared to unmodified PLGA may depend on the end application.

Taken together, this provides a simple one-step strategy to pre-functionalize polyester polymers to produce biomaterials with enhanced cell surface attachments under physiological conditions. This strategy eliminates the requirement for laborious and extensive post-functionalization processes. The PLGA_{DMAE} MPs presented in this work readily attach to a clinically relevant cell type, ADSCs, maintain a low toxicity and display a more uniform degradation profile than PLGA.

Thus, a new strategy to pre-functionalize hydrolytically degradable polyester polymers with cell adhesive moieties is presented that addresses at least some of the shortcomings of these polymers for advanced biomedical applications. The pre-functionalization method has been validated by synthesizing PLGA polymers with DMAE groups by using a ROP technique. The introduction of the DMAE groups into the PLGA polymers did not change the bulk or thermal properties of the polymers and the successful fabrication of MPs via a membrane emulsion technique was demonstrated. Furthermore, it was shown that the clinically relevant ADSCs more readily attach to the surface of these MPs under physiological conditions, than to the unmodified PLGA MPs. The introduction of the DMAE group into the polymers elicited a faster and more uniform degradation of the polymer. Moreover, the PLGA_{DMAE} MPs were shown to maintain the already desirable low cytotoxicity profile associated with PLGA MPs. This data set validates the pre-functionalization strategy and could be widely used to develop other hydrolytically degradable polyesters with a broad range of advanced biomedical applications.

### Additional Experimental Results

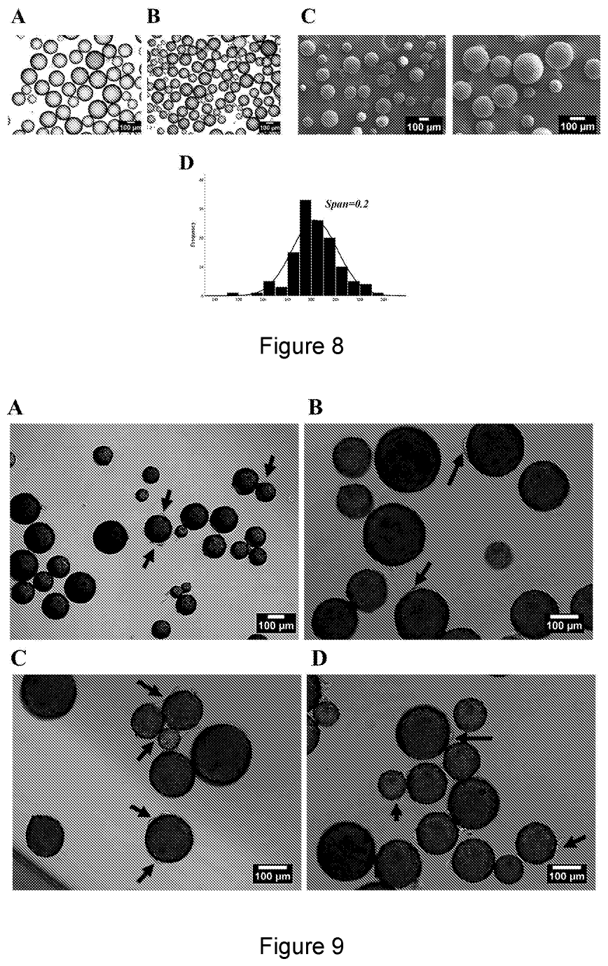

Further PLGA_{DMAE} microparticles were prepared by a membrane emulsification technique similar to that described above except that the solution of PLGA_{DMAE} polymer in DCM used as the organic phase was 15% w/v PLGA_{DMAE} polymer in 14 mL DCM (rather than 12.5% w/v) and the organic phase was injected via a stainless-steel hydrophilic ring membrane (100 μm pore size) into the continuous phase (130 mL) at a rate of 1 mL/min and a pre-adjusted stirring speed of 600 rpm. Additionally, the microparticles were left to sediment at room temperature in microcentrifuge tubes. This produced larger PLGA_{DMAE} microparticles. FIG. 8 shows the morphology and size distribution analysis of these PLGA_{DMAE} microparticles.

ADSCs were then attached to these PLGA_{DMAE} microparticles using the cell attachment process (Method 2) described above. Phase contrast images (FIG. 9) show the ADSCs attached to the PLGA_{DMAE} microparticles 24 hours after culturing. The ADSCs were cultured following the protocol described above.

While the invention has been described with reference to certain examples, those skilled in the art will appreciate that various modifications, changes, omissions, and substitutions can be made without departing from the spirit of the disclosure. It is intended, therefore, that the invention be limited by the scope of the following claims and their equivalents. Unless otherwise stated, the features of any dependent claim can be combined with the features of any of the other dependent claims and any of the independent claims.

1. A biodegradable polymer having the following formula: 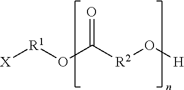
wherein
X is selected from NH₂, NHR³, NR³₂ and NR³₃⁺;
R¹ is an alkylene group;
each R² is independently selected from a —(CR⁴₂)ₘ— group or a —(CR⁴₂)ₒO(CR⁴₂)ₚ— group;
each R³ is independently an alkyl group;
each R⁴ is independently selected from hydrogen and an alkyl group; and
n, m, o and p are independently 1 or more.

2. The biodegradable polymer of claim 1, wherein R¹ is a C1 to C20 alkylene group.

3. The biodegradable polymer of any preceding claim, wherein each R³ is independently selected from C1 to C20 alkyl, for example, methyl, ethyl, propyl, or butyl.

4. The biodegradable polymer of any preceding claim, wherein each R⁴ is independently selected from hydrogen and a C1 to C20 alkyl group; and wherein m is selected from 1 to 20.

5. The biodegradable polymer of any preceding claim, wherein R¹ is a C1 to C5 alkylene group; each R³ is independently a C1 to C5 alkyl group; each R⁴ is independently hydrogen or a C1 to C5 alkyl group; and m is selected from 1 to 5.

6. The biodegradable polymer of any preceding claim comprising an XR¹O-terminated poly(lactic acid), XR¹O-terminated poly(glycolic acid), XR¹O-terminated poly(lactic-co-glycolic acid), XR¹O-terminated poly(caprolactone), XR¹O-terminated poly(hydroxybutyrate), XR¹O-terminated poly(p-dioxanone), or XR¹O-terminated poly(3-hydroxyvalerate).

7. The biodegradable polymer of any preceding claim, wherein the polymer has a weight average molecular weight (M_{w}) of from 1000 to 1 million, for example, 10,000 to 200,000, 10,000 to 200,000, 10,000 to 50,000, 50,000 to 200,000 or 200,000 to 1 million.

8. The biodegradable polymer of any preceding claim, wherein the polymer has a number average molecular weight (Mₙ) of from 500 to 1 million, for example, 15,000 to 35,000.

9. The biodegradable polymer of any preceding claim wherein the polymer has a dispersity (M_{w}/Mₙ) of from 1 to 4, for example, 1 to 2 or 2 to 4.

10. Microparticles comprising the biodegradable polymer of any preceding claim.

11. The microparticles of claim 11, wherein the microparticles have an average diameter of 2 mm or less, for example, 10 nm to 2 mm, 10 nm to 1 μm, 10 nm to 100 nm, 1 μm to 2 mm, 50 μm to 100 μm, 1 μm to 20 μm, 20 μm to 50 μm, 50 μm to 100 μm.

12. A method comprising:
combining an amino alcohol with a cyclic ester and performing ring-opening polymerisation to form a biodegradable polymer;
wherein the amino alcohol has the formula XR¹OH; and
wherein
the cyclic ester is selected from cyclic monoesters having the formula (1), cyclic diesters having the formula (2), and combinations thereof; 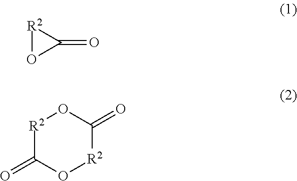
X is selected from NH₂, NHR³, NR³₂ and NR³₃⁺;
R¹ is an alkylene group;
each R² is independently selected from a —(CR⁴₂)ₘ— group or a —(CR⁴₂)ₒO(CR⁴₂)ₚ— group;
each R³ is independently an alkyl group;
each R⁴ is independently selected from hydrogen and an alkyl group; and
m, o and p are independently 1 or more.

13. The method of claim 12, wherein the cyclic ester is selected from lactide, glycolide, caprolactone, and combinations thereof.

14. The method of any of claims 12 to 13, wherein the ring-opening polymerisation is catalysed, for example, by a metal catalyst, such as tin(II) 2-ethylhexanoate, 4-dimethylaminopyridine (DMAP), or aluminium isopropoxide.

15. The method of any of claims 12 to 14, further comprising forming microparticles of the biodegradable polymer; electrospinning the biodegradable polymer; extruding the biodegradable polymer; moulding the biodegradable polymer; or three-dimensional printing of the biodegradable polymer, for example, using stereolithography or photopolymerisation by digital light processing or UV laser processing.

16. The method of any of claims 12 to 15, further comprising culturing cells in the presence of the biodegradable polymer to form a cell-coated biodegradable polymer.

17. A method of culturing cells comprising combining the biodegradable polymer of any of claims 1 to 9 or the microparticles of any of claims 10 to 11 with a cell culture medium and incubating the composition.

18. A composition comprising cells supported on the biodegradable polymer of any of claims 1 to 9 or on the microparticles of any of claims 10 to 11, wherein the composition may be a supported tissue and/or wherein the cells may comprise stem cells (e.g., embryonic stem cells, tissue-specific stem cells, mesenchymal stem cells, hematopoietic stem cells, or induced pluripotent stem cells), β cells, antibody generating cells, chimeric immunoreceptor T cells (CAR T cells), or specialised cells (e.g., bone cells, skin cells, muscle cells, cardiac cells, lung cells, or intestinal cells) or a mixture thereof.

19. A tissue scaffold, a microcarrier for of biologics (e.g., genetic material, drugs, cells or antibodies), or a medical implant comprising the biodegradable polymer of any of claims 1 to 9 or the microparticles of any of claims 10 to 11.

20. Use of the biodegradable polymer of any of claims 1 to 9 or the microparticles of any of claims 10 to 11 in tissue engineering or regenerative medicine.
